(19) United States
(12) Patent Application Publication
(10) Pub. No.: US 2016/0346432 A1
(43) Pub. Date: Dec. 1, 2016

(54) SHEET-FORM CELL GROWTH SCAFFOLD PARTICLES AND GRAFTS, AND METHODS FOR SAME

(71) Applicant: Cook Regentec LLC, Indianapolis, IN (US)

(72) Inventors: Shelly J. Zacharias, Indianapolis, IN (US); Steven Charlebois, West Lafayette, IN (US); Charles Leland Baxter, West Lafayette, IN (US)

(21) Appl. No.: 15/167,061

(22) Filed: May 27, 2016

Related U.S. Application Data
(60) Provisional application No. 62/167,263, filed on May 27, 2015.

(51) Int. Cl. A61L 27/36; A61L 27/24; A61L 27/50

(52) U.S. Cl. CPC A61L 27/3691; A61L 27/50; A61L 27/3604; A61L 27/24; A61L 27/3629; A61L 2400/06; A61L 2300/426; A61L 2300/252

(57) ABSTRACT

Described are sheet-form cell growth scaffold particles, and methods for preparing and using them. The particles can be prepared using punch or other cutting operations to provide relatively uniform populations of particles in terms of shape and size, desirably employing a stack of multiple sheets of starting material and multiple punches. Cellularized grafts and/or cell conditioned media can be prepared using the sheet-form cell growth scaffold particles.

### REFERENCE TO RELATED APPLICATION

This application claims the benefit of U.S. Provisional Application No. 62/167,263, filed May 27, 2015, which is hereby incorporated by reference.

### BACKGROUND

Aspects of the present invention relate to biologics-based materials and methods, and in specific aspects relates to medical grafts, in some forms containing cells, and to materials and methods for their preparation or use.

Implantable graft materials including extracellular matrices and/or viable cells are known. In certain practices, cells to be introduced into the patient can be combined with a substrate to form a cell-containing implantable graft. Sometimes, these uses involve a culture period in which the number of cells is expanded after application to the scaffold material. Other modes of use do not involve such expansion. Rather, the cells are applied to the substrate and implanted without expansion of the number of cells. In other practices, a medium in which cells have been cultured is separated from the cells and then administered to the patient. Such a “cell-conditioned” medium contains biologic substances produced and secreted by the cells into the medium, which may have therapeutic benefit. Still further, in other forms, extracellular matrix grafts are administered to patients without added cells.

Despite demonstrated promise, the clinical implementation of biologics-based medical technology has been relatively slow. Needs exists for improved and/or alternative materials and methods that are useful in the practice of biologics-based medical or research technology. In certain of its aspects, the present invention is addressed to these needs.

### SUMMARY

Certain aspects of the present invention relate to sheet-form cell growth scaffold particles, methods for their preparation and use, and compositions including them. According to one embodiment, provided is a method for preparing cell growth scaffold particles. The method includes forcing at least one punch through at least one sheet of cell growth scaffold material to remove from the sheet a sheet-form scaffold particle, and collecting the sheet-form scaffold particle removed from the sheet in the forcing step. Such a method can also include applying tension to the at least one sheet during the forcing, and the tension can be applied by pressing a resilient member against the at least one sheet. Such pressing can occur during the forcing, and can be released during movement of the punch to withdraw the punch from the at least one sheet. The resilient member can comprises a resilient tubular wall having a leading end defining a perimeter, and the pressing can include pressing the leading end of the tubular wall against the at least one layer. In preferred forms, such methods include using multiple punches, such as two to twenty punches, to simultaneously punch through multiple sheets of cell growth scaffold material, such as two to ten sheets. In addition or alternatively, the punch(es) can create a pattern of spaced holes in the starting sheet(s), with the holes spaced from one another so that an integral punched remnant of the sheet remains.

In another embodiment, provided is a particulate cell growth scaffold composition that includes a population of sheet-form cell growth scaffold particles, wherein the particles have perimeters defined by cut edges. The cut edges are preferably mechanically cut edges, and can be free from heat denatured collagen and present exposed cut ends of collagen fibers. Preferred cell growth scaffold particles include an extracellular matrix tissue material, and preferably wherein the tissue material retains one or more bioactive agents native to the source tissue of the extracellular matrix tissue material, and more preferably wherein the one or more bioactive agents includes basic fibroblast growth factor (FGF-2), transforming growth factor beta (TGF-beta), epidermal growth factor (EGF), cartilage derived growth factor (CDGF), platelet derived growth factor (PDGF), glycoproteins, proteoglycans, and/or glycosaminoglycans. Compositions are also provided that include such scaffold particles and cells.

Provided in another embodiment is a method for preparing a composition that includes incubating cells in suspension in the presence of a composition including sheet-form cell growth scaffold particles as describe herein. The incubating can include culturing the cells sufficiently to form cellularized bodies in which the cells have deposited extracellular matrix proteins endogenous to the cells in and/or on the sheet-form scaffold particles. In some forms, the culturing is sufficiently conducted so that at least 1% of the collagen in said cellularized bodies is endogenous to the cells. In some forms, the method also includes detaching the cells from the scaffold particles or cellularized bodies, for example to form a single cell suspension of the cells. Additionally or alternatively, the method can also include collecting a liquid medium which has been conditioned during the culturing, to provide a “cell conditioned medium” that can be put to therapeutic use.

In still other embodiments, provided are methods for treating a patient that include administering to the patient sheet-form cell growth scaffold particles as described herein, cellular grafts as described herein, or conditioned medium as described herein.

Additional embodiments, as well as features and advantages thereof, will be apparent from the descriptions herein.

### BRIEF DESCRIPTION OF THE FIGURES

FIG. 1 provides a digital image of an illustrative embodiment of a sheet-form cell growth scaffold particle.

FIG. 2 provides an illustration of a punch arrangement for preparing sheet-form cell growth scaffold particles.

FIG. 3 provides an illustration of another punch arrangement for preparing sheet-form cell growth scaffold particles.

FIG. 4 provides an illustration of an illustrative embodiment of a cellular graft composition.

FIG. 5 provides a digital image showing day 11 Canine URCs attached to an ECM disc particle, Calcien AM live-dead stained, as described in the Experimental below.

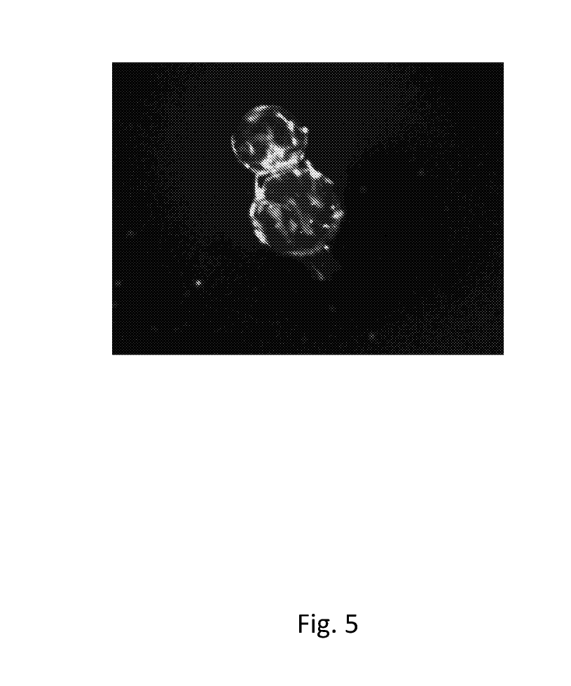

FIG. 6 shows graphs representing cytokine analysis for MCP-1, KC-Like and IL-8, evaluated from media alone, ECM disc particles alone or cells cultured on SIS disc particles, as described in the Experimental below.

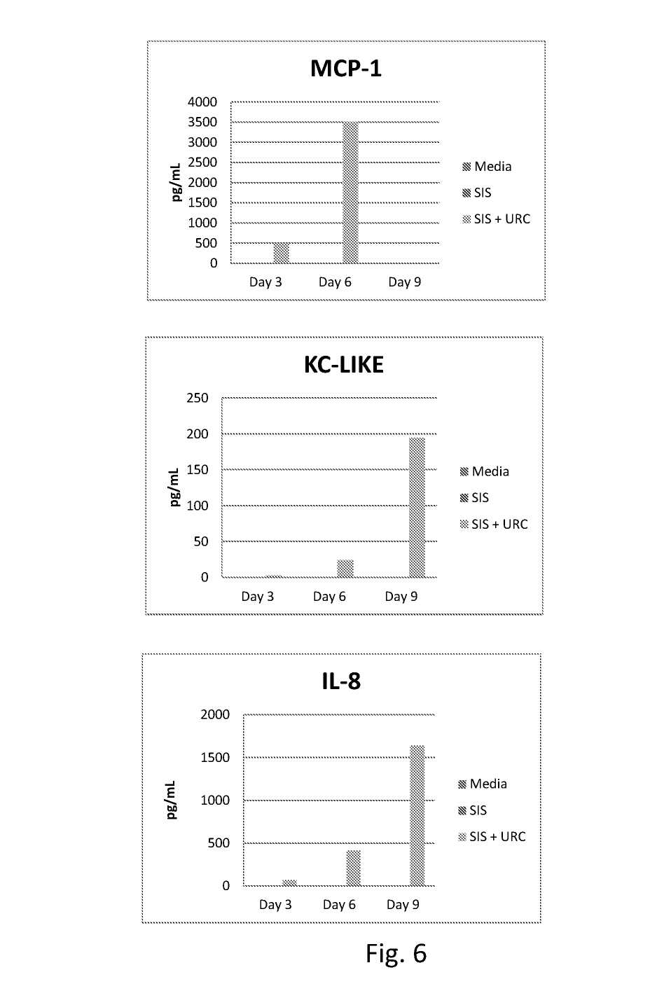

FIG. 7 provides digital images demonstrating an ability of ECM disc particles to preserve and protect cells on injection, as demonstrated by 10-million RFP-HeLa cells+injectable ECM disc particles, IVIS Lumina imaged (a) in a 1 cc syringe with a 23 G needle at Day 0; (b) 100 μikers injected intra-muscularly into NOD SCID mouse and imaged after approximately 48-hours, as described in the Experimental below.

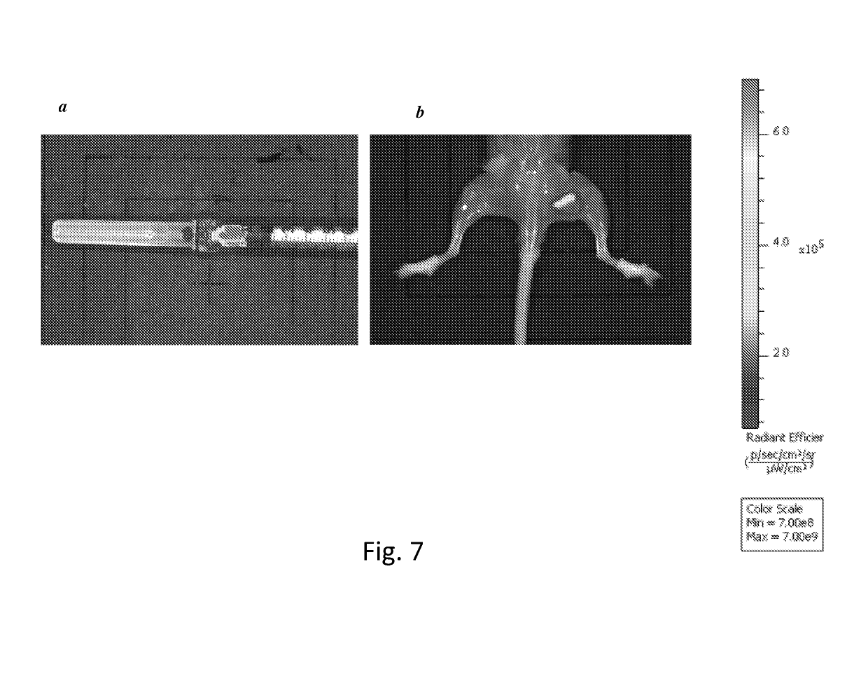

### DETAILED DESCRIPTION

For the purpose of promoting an understanding of the principles of the invention, reference will now be made to embodiments, some of which are illustrated with reference to the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Additionally, in the detailed description below, numerous alternatives are given for various features related to the composition or size of materials, or to modes of carrying out methods. It will be understood that each such disclosed alternative, or combinations of such disclosed alternatives, can be combined with the more generalized features discussed in the Summary above, or set forth in the Claims below, to provide additional disclosed embodiments herein.

As disclosed above, aspects of the present invention relate to materials and methods that are useful for example in practices related to medicine or research. In certain embodiments provided are sheet-form cell growth scaffold particles, and methods of their preparation and use, for example their use in making cellularized compositions that can be used as tissue grafts and their use in making cell conditioned media that can be used beneficially in therapies.

In some embodiment herein, the sheet-form cell growth scaffold particles can have a maximum cross sectional dimension of about 20 microns to about 2000 microns, or about 100 to about 1000 microns, or about 100 to 500 microns. The sheet-form scaffold particles can be substantially uniform in size relative to one another, e.g. having maximum cross sectional dimensions within about 20%, or 10%, of one another, or can vary in size with respect to one another (e.g. having some smaller particles and some larger particles, potentially a controlled overall population created by mixing two or more substantially uniform particle populations, where the populations are of different sizes relative to one another). In advantageous forms, the particles are in sheet form, and can have a sheet thickness of about 20 to about 1000 microns, or about 20 to about 500 microns, or about 20 to about 300 microns. Additionally or alternatively, the sheet-form particles can have maximum cross sectional axis length considered in the plane of the sheet (e.g. height or width) that is greater than the sheet thickness. The sheet-form scaffold particles can have shapes that are regular with respect to one another or which are irregular with respect to one another. In certain embodiments, the sheet-form scaffold particles can have a perimeter edge defined by a continuous curve (e.g. as in a generally circular or ovoid or annular (e.g. “washer”) shaped sheet particle), and in other forms can have a polygonal perimeter edge (e.g. having three to ten sides, e.g. triangular, square or otherwise rectangular, pentagonal, hexagonal, star, etc.) shape. For example, the scaffold particles, or a substantial percentage of them in the composition (e.g. above about 25%), when considered in the plane of the sheet, can have a maximum cross sectional dimension axis which is no more than about two times the length of the cross sectional dimension axis taken on a line perpendicular to and centered on the maximum cross sectional dimension axis; preferably, at least about 50% of the substrate particles will have this feature, and more preferably at least about 70% of the substrate particles will have this feature. Such particulate scaffold materials constitute an embodiment of the present invention, alone (e.g. as cell-free tissue graft materials) or used in combination with cells as discussed herein.

Small, sheet-form cell growth scaffold particles as discussed above can be cut from larger sheets of cell growth scaffold material. In certain embodiments, the larger sheet of material will be an extracellular matrix sheet material harvested from a tissue source and decellularized, as discussed herein. Sheet-form particles having the above-described characteristics are in certain embodiments mechanically cut from larger ECM sheets using mechanical implements such as punches and/or dies. In desired embodiments, the cutting method used will not eliminate the native bioactive ECM character or native bioactive ECM molecules, as discussed in more detail herein, when this character or these molecules are resident in a larger starting ECM sheet being processed. Additionally, the ECM sheet being processed, and the resultant ECM sheet particles can have a retained native epithelial basement membrane on one or both sides of the sheet material, and/or biosynthetically deposited basement membrane components on one or both sides of the sheet. To prepare particles with biosynthetically deposited non-native basement membrane components, a decellularized ECM sheet can be conditioned by growing epithelial cells, endothelial cells, stem cells, or other cells on one or both sides of the sheet to deposit basement membrane components. The cells can then be removed while leaving the basement membrane components, and the sheet then processed to prepare the sheet-form particles as described herein.

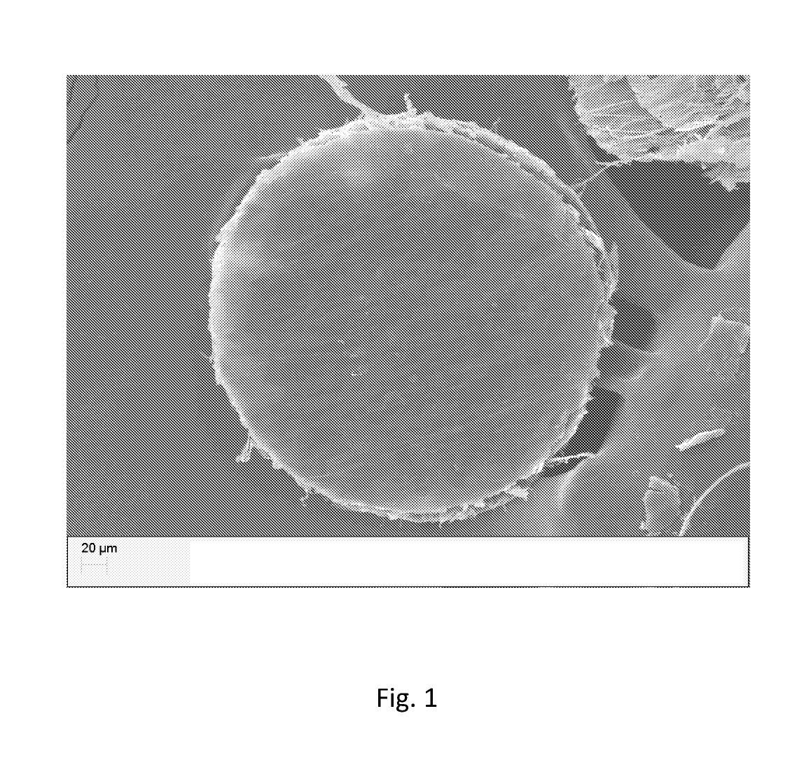

FIG. 1 provides a digital image of an illustrative, small ECM disc that was cut with a punch from a larger ECM sheet. As can be seen, the illustrated sheet-form scaffold particle is generally circular in shape and has a diameter of about 250 microns. As well, the sheet form scaffold particle has a cut perimeter edge presenting exposed cut ends of collagen fibers, which can be beneficial to cell attachment to the particles. When such particles are cut using a mechanical cutting implement such as a punch or punch and die while avoiding significant generation of heat through friction or otherwise, the cut perimeter edge can in some embodiments be free of or essentially free of heat-denatured collagen. Similarly, sheet-form particles cut from other fibrous scaffold sheet materials can have exposed cut ends of fibers from which the sheets are formed.

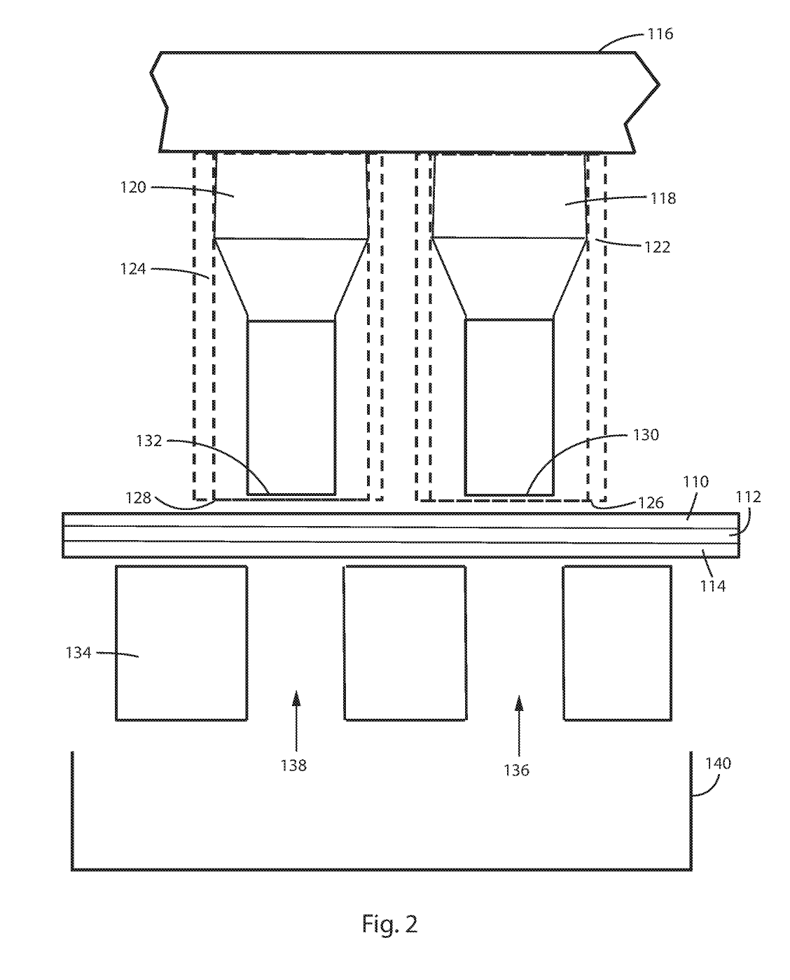

With reference now to FIG. 2, shown is an illustrative embodiment of an arrangement for creating sheet-form scaffold particles using a punch and die system. In particular, shown is a stack of three sheets 110, 112 and 114 of cell growth scaffold material. As discussed herein, the sheets 110, 112 and 114 generally lie in the X-Y axis (X axis is left to right, and Y axis is into and out of the page, in FIG. 2), whereas the Z axis is perpendicular to the plane of the sheets (up and down in FIG. 2). While the illustrated arrangement includes three sheets of cell growth scaffolding material, it will be understood that other numbers of sheets can be used, including one sheet, two or more sheets, or in certain forms two to ten sheets. A punch head 116 includes a plurality of punches, such as two punches 118 and 120. In other embodiments, for example two to twenty punches like punches 118 and 120 can be used in such an arrangement. Fitted around punches 118 and 120 are resilient sleeves or tubes 122 and 124 (shown in dotted lines). Sleeves 122 and 124 have respective distal ends 126 and 128, which extend beyond the leading ends 130 and 132 of punches 118 and 120. Situated below the stack of ECM sheets 110, 112 and 114 is a die piece 134 having first hole 136 and second hole 138 sized to receive a portion of punches 118 and 120, respectively, in a punch and die cutting operation. In use, punch head 116 is directed toward the stack of ECM sheets 110, 112 and 114 (in the Z axis) causing resilient sleeves 122 and 124 to press into the stack prior to contact by the punch leading ends 130 and 132. In this manner, sleeves 122 and 124 can stabilize and preferably apply tension to the region of sheets 110, 112 and 114 to be cut out by punches 118 and 120 and their respective die holes 136 and 138. Continued movement of the punch head 116 in the direction of the stack of sheets 110, 112 and 114 (in the Z axis) causes punches 118 and 120 to press into the sheets 110, 112 and 114 and continue into holes 136 and 138 of die 134, causing sheet-form scaffold particles to be severed from sheets 110, 112 and 114. The sheet-form scaffold particles, after separation from the sheets 110, 112, and 114, can pass through the holes 136 and 138 (e.g. aided by the force of gravity) and be collected in a collection container 140, such as a vial or other chamber. In the illustrated embodiment, punches 118 and 120 and their respective holes 136 and 138 are generally circular, resulting in the formation of generally circular sheet-form scaffold particles. As discussed above, it will be understood that other regular shapes can be formed using punches and optionally dies with die holes of corresponding shape. Where the punching operation involves moving the punch head 116 in the X and/or Y axis, the die 134 can be moved in registry with the punch head 116 to maintain alignment of the punches 118 and 120 and their respective die holes 136 and 138; alternatively, a stationary die 134 could be provided with more holes than there are punches on punch head 116, and the punch head 116 can be moved in the X and/or Y axis to position its punches over a new set of holes in the die each time it is moved. As well, it will be understood that in other operations the punch head 116 and the die 134 can be held stationary in the X and Y axes, and the stack of sheets 110, 112 and 114 moved in the X and/or Y axis in between punching strokes in order to punch new regions of the sheets 110, 112 and 114. It will be understood that in preferred embodiments, sheets 110, 112 and 114 (or any number of sheets present) are not bonded or otherwise adhered to one another. In this fashion, the sheet-form particles created by the punching operation from the respective sheets 110, 112 and 114 readily separate from one another during and/or after the punching operation. In other embodiments, some or all of the sheets in a stack can be bonded to one another, resulting in the formation of multilaminate sheet-form cell growth scaffold particles.

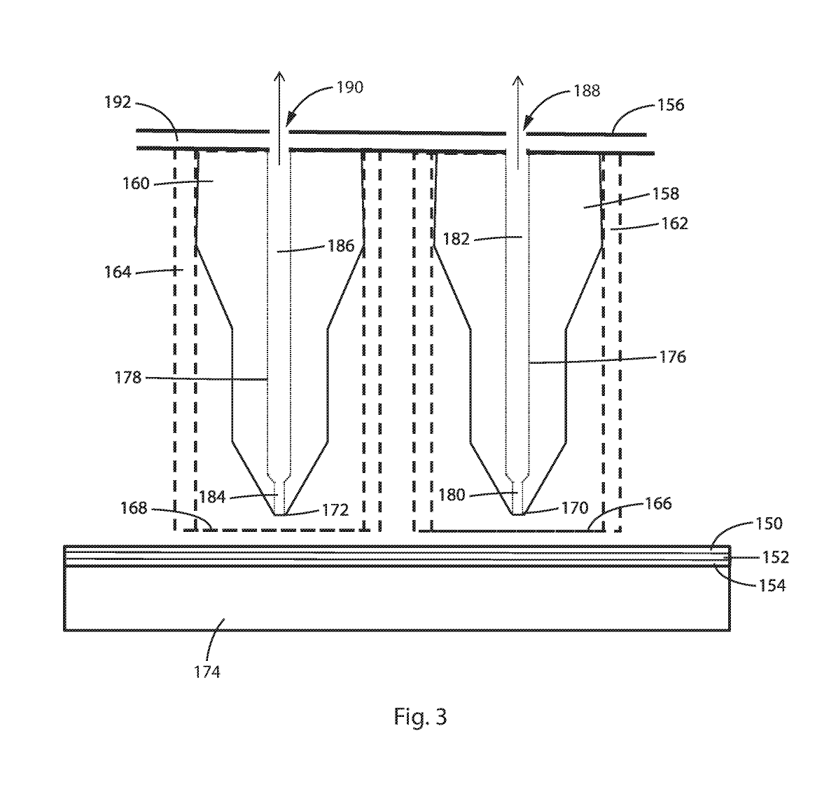

In addition to punching operations as described above, it will be understood that other punching or cutting operations can also be used to create sheet-form scaffold particles. For example, with reference to FIG. 3, shown is another illustrative arrangement for creating sheet-form scaffold particles using a punch system. In particular, shown again is the stack of three sheets 150, 152 and 154 of cell growth scaffold material. Again, while the illustrated arrangement includes three sheets of cell growth scaffolding material, it will be understood that other numbers of sheets can be used, including one sheet, two or more sheets, or in certain forms two to ten sheets. A punch head 156 includes a plurality of punches, such as two punches 158 and 160. In other embodiments, for example two to twenty punches like punches 158 and 160 can be used in such an arrangement. Fitted around punches 158 and 160 are resilient sleeves or tubes 162 and 164 (shown in dotted lines). Sleeves 162 and 164 have respective distal ends 166 and 168, which extend beyond the leading ends 170 and 172 of punches 158 and 160. Situated below the stack of ECM sheets 150, 152 and 154 is a punch backing 174. Punch backing 174 is sufficiently compliant to avoid damage to the punches, but sufficiently tough that pieces of the backing are not cut out by the punches. Punches 158 and 160 have respective passages 176 and 178 extending longitudinally through them. Passage 176 has a first portion 180 extending from leading end 170 and having a first diameter, and a second portion 182 having a second diameter, where the second diameter is larger than the first diameter. Similarly, passage 178 has a first portion 184 extending from leading end 172 and having a first diameter, and a second portion 186 having a second diameter, where the second diameter is larger than the first diameter. First portions 180 and 184 have a diameter corresponding to the diameter of the sheet-form particles to be formed. Passages 176 and 178 fluidly communicate with openings 188 and 190 in a wall 192 of punch head 156. In use, punch head 156 is directed toward the stack of ECM sheets 150, 152 and 154 causing resilient sleeves 162 and 164 to press into the stack prior to contact by the punch leading ends 170 and 172. In this manner, sleeves 162 and 164 can stabilize and preferably apply tension to the region of sheets 150, 152 and 154 to be cut out by punches 158 and 160. Continued movement of the punch head 156 in the direction of the stack of sheets 110, 112 and 114 causes punches 158 and 160 to press into and cut the sheets 150, 152 and 154, causing sheet-form scaffold particles to be severed from sheets 150, 152 and 154. The sheet-form scaffold particles are collected in passages 176 and 178 during the punch operation, first within first portions 180 and 184 and after these are filled during multiple punch strokes within portions 182 and 186. With sufficient numbers of punch strokes, passages 176 and 178 become filled with the sheet-form particles, after which continued punching forces the uppermost particles through openings 188 and 190, which can be collected in a chamber in the punch head 156 or otherwise. If desired, the collection of the particles in and through passage 176 and 178 can be aided by the application of a vacuum to the passages 176 and 178 to draw the particles toward and potentially into the punch head. In other embodiments where punches have internal passages such as passages 176 and 178, or passages having a consistent size throughout the punch, after particles have been collected in the passages through one or more punching strokes, a push rod can be forced through the passages in a direction from the punch head 156 to the leading ends 172 and 178, for example in an automated operation, to eject the particles from the passages and out of the leading ends 172 and 178. Such ejected particles can, for example, be ejected into a vial, bin or other chamber for collection.

Punching operations with the arrangement shown in FIG. 3 can involve moving the punch head 156 in the X and/or Y axis, and in the Z axis during a downward punching stroke; alternatively, punch head 156 can be held stationary in the X and Y axes, and the stack of sheets 150, 152 and 154 and backing 174 moved in the X and/or Y axes in between punching strokes in the Z axis order to punch new regions of the sheets 110, 112 and 114. Again, it will be understood that in preferred embodiments, sheets 150, 152 and 154 (or any number of sheets present) are not bonded or otherwise adhered to one another. In this fashion, the sheet-form particles created by the punching operation from the respective sheets 150, 152 and 154 readily separate from one another during and/or after the punching operation. In other embodiments, some or all of the sheets in a stack can be bonded to one another, resulting in the formation of multilaminate sheet-form cell growth scaffold particles.

Punch operations to prepare sheet-form scaffold particles as described herein are preferably conducted in automated fashion using computerized numerical control (CNC) to move and operate the punch head, die, stack of sheets, and/or punch backing, as appropriate. Multiple electrically powered linear actuators can be used under CNC control to achieve the operations needed for punching. In preferred operations, at least about 50% punch efficiency is achieved (meaning that at least about 40% by weight of the original sheet(s) subjected to the punching operation is recovered as the sheet-form scaffold particles), typically in the range of 40% to 60%, and preferably in the range of 50% to 60%. The punches are preferably made of tungsten carbide or another similarly hard metal.

While punching arrangements and operations have been described in connection with FIGS. 2 and 3 above, it will be understood that other suitable mechanical cutting and other cutting operations suitable for the preparation of sheet-form scaffold particles will be apparent to those of skill in the art from the descriptions herein.

As noted above, sheet materials used to prepare sheet-form scaffold particles can comprise extracellular matrix (ECM) tissue. The ECM tissue can be obtained from a warm-blooded vertebrate animal, such as an ovine, bovine or porcine animal. For example, suitable ECM tissue include those comprising submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa including small intestinal submucosa, stomach submucosa, urinary bladder submucosa, and uterine submucosa. ECM tissues comprising submucosa (potentially along with other associated tissues) useful in the present invention can be obtained by harvesting such tissue sources and delaminating the submucosa-containing matrix from smooth muscle layers, mucosal layers, and/or other layers occurring in the tissue source. Porcine tissue sources are preferred sources from which to harvest ECM tissues, including submucosa-containing ECM tissues.

ECM tissue when used in the invention is preferably decellularized and highly purified, for example, as described in U.S. Pat. No. 6,206,931 to Cook et al. or U.S. Patent Application Publication No. US2008286268 dated Nov. 20, 2008, publishing U.S. patent application Ser. No. 12/178,321 filed Jul. 23, 2008, all of which are hereby incorporated herein by reference in their entirety. Preferred ECM tissue material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 or U.S. Patent Application Publication No. US2008286268 may be characteristic of any ECM tissue used in the present invention.

In certain embodiments, the ECM tissue material used as or in the sheet material will be a membranous tissue with a sheet structure as isolated from the tissue source. The ECM tissue can, as isolated, have a layer thickness that ranges from about 50 to about 250 microns when fully hydrated, more typically from about 50 to about 200 microns when fully hydrated, although isolated layers having other thicknesses may also be obtained and used. These layer thicknesses may vary with the type and age of the animal used as the tissue source. As well, these layer thicknesses may vary with the source of the tissue obtained from the animal source.

The ECM tissue material utilized desirably retains a structural microarchitecture from the source tissue, including structural fiber proteins such as collagen and/or elastin that are non-randomly oriented. Such non-random collagen and/or other structural protein fibers can in certain embodiments provide an ECM tissue that is non-isotropic in regard to tensile strength, thus having a tensile strength in one direction that differs from the tensile strength in at least one other direction.

The ECM tissue material may include one or more bioactive agents native to the source of the ECM tissue material and retained in the ECM tissue material through processing. For example, a submucosa or other remodelable ECM tissue material may retain one or more native growth factors such as but not limited to basic fibroblast growth factor (FGF-2), transforming growth factor beta (TGF-beta), epidermal growth factor (EGF), cartilage derived growth factor (CDGF), and/or platelet derived growth factor (PDGF). As well, submucosa or other ECM materials when used in the invention may retain other native bioactive agents such as but not limited to proteins, glycoproteins, proteoglycans, and glycosaminoglycans. For example, ECM materials may include heparin, heparin sulfate, hyaluronic acid, fibronectin, cytokines, and the like. Thus, generally speaking, a submucosa or other ECM material may retain from the source tissue one or more bioactive components that induce, directly or indirectly, a cellular response such as a change in cell morphology, proliferation, growth, protein or gene expression.

Submucosa-containing or other ECM materials used in the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with appropriate staining. Such non-collagenous solids can constitute a significant percentage of the dry weight of the ECM material in certain inventive embodiments, for example at least about 1%, at least about 3%, and at least about 5% by weight in various embodiments of the invention.

The submucosa-containing or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the formation of new blood vessels into the materials. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of such native bioactive components, non-native bioactive components such as those synthetically produced by recombinant technology or other methods (e.g., genetic material such as DNA), may be incorporated into an ECM material used in the invention. These non-native bioactive components may be naturally-derived or recombinantly produced proteins that correspond to those natively occurring in an ECM tissue, but perhaps of a different species. These non-native bioactive components may also be drug substances. Illustrative drug substances that may be added to materials include, for example, anti-clotting agents, e.g. heparin, antibiotics, anti-inflammatory agents, thrombus-promoting substances such as blood clotting factors, e.g., thrombin, fibrinogen, and the like, and anti-proliferative agents, e.g. taxol derivatives such as paclitaxel. Such non-native bioactive components can be incorporated into and/or onto ECM material in any suitable manner, for example, by surface treatment (e.g., spraying) and/or impregnation (e.g., soaking), just to name a few. Also, these substances may be applied to the ECM material in a premanufacturing step, immediately prior to the procedure (e.g., by soaking the material in a solution containing a suitable antibiotic such as cefazolin), or during or after engraftment of the material in the patient.

Inventive graft compositions herein can incorporate xenograft ECM material (i.e., cross-species material, such as tissue material from a non-human donor to a human recipient), allograft ECM material (i.e., interspecies material, with tissue material from a donor of the same species as the recipient), and/or autograft ECM material (i.e., where the donor and the recipient are the same individual). Further, any exogenous bioactive substances incorporated into an ECM material may be from the same species of animal from which the ECM material was derived (e.g. autologous or allogenic relative to the ECM material) or may be from a different species from the ECM material source (xenogenic relative to the ECM material). In certain embodiments, ECM tissue material will be xenogenic relative to the patient receiving the graft, and any added cells or other exogenous material(s) will be from the same species (e.g. autologous or allogenic) as the patient receiving the graft. Illustratively, human patients may be treated with xenogenic ECM materials (e.g. porcine-, bovine- or ovine-derived) that have been modified with exogenous human cells and/or serum proteins and/or other material(s) as described herein, those exogenous materials being naturally derived and/or recombinantly produced.

When used in the invention, ECM materials can be free or essentially free of additional, non-native crosslinking, or may contain additional crosslinking. Such additional crosslinking may be achieved by photo-crosslinking techniques, by chemical crosslinkers, or by protein crosslinking induced by dehydration or other means. However, because certain crosslinking techniques, certain crosslinking agents, and/or certain degrees of crosslinking can destroy the remodelable properties of a remodelable material, where preservation of remodelable properties is desired, any crosslinking of the remodelable ECM material can be performed to an extent or in a fashion that allows the material to retain at least a portion of its remodelable properties. Chemical crosslinkers that may be used include for example aldehydes such as glutaraldehydes, diimides such as carbodiimides, e.g., 1-ethyl-3-(3-dimethylaminopropyl)carbodiimide hydrochloride, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

In addition to or as an alternative to ECM materials, the scaffold material used in the invention may be comprised of other suitable materials. Illustrative materials include, for example, synthetically-produced substrates comprised or natural or synthetic polymers. Illustrative synthetic polymers can include nonresorbable synthetic biocompatible polymers, such as cellulose acetate, cellulose nitrate, silicone, polyethylene teraphthalate, polyurethane, polyamide, polyester, polyorthoester, polyanhydride, polyether sulfone, polycarbonate, polypropylene, high molecular weight polyethylene, polytetrafluoroethylene, or mixtures or copolymers thereof; or resorbable synthetic polymer materials such as polylactic acid, polyglycolic acid or copolymers thereof, polyanhydride, polycaprolactone, polyhydroxy-butyrate valerate, polyhydroxyalkanoate, or another biodegradable polymer or mixture thereof. Preferred scaffold materials comprised of these or other materials will be porous matrix materials configured to allow cellular invasion and ingrowth into the matrix.

In preferred modes, the sheet or sheets of cell growth scaffold material are in a dried condition during the punching or other cutting operation. For example, an extracellular matrix tissue material or other material as described herein can be lyophilized, air dried, oven dried, vacuum dried, or otherwise dried, to provide a starting material for the punching or cutting operation. In some embodiments, the extracellular matrix tissue material or other sheet material can have a water content of less than about 15% by weight, or less than about 10% by weight, during the punching/cutting operation.

In certain forms, the sheet form scaffold material used in the invention can be treated with a cell culture medium and/or blood or a blood fraction, prior to contact with cells. For example, a sheet of cell growth scaffold material used to prepare sheet-form cell growth scaffold particles as described herein can be pre-treated with a cell culture medium and/or blood or a blood fraction, and can incorporate such substance(s) during the punch or other cutting operation (e.g. as dried into a sheet of scaffolding material that is then punched or cut in dried condition) to create the sheet-form particles. In addition or alternatively, formed sheet-form particles can be treated with such substances prior to contact with cells.

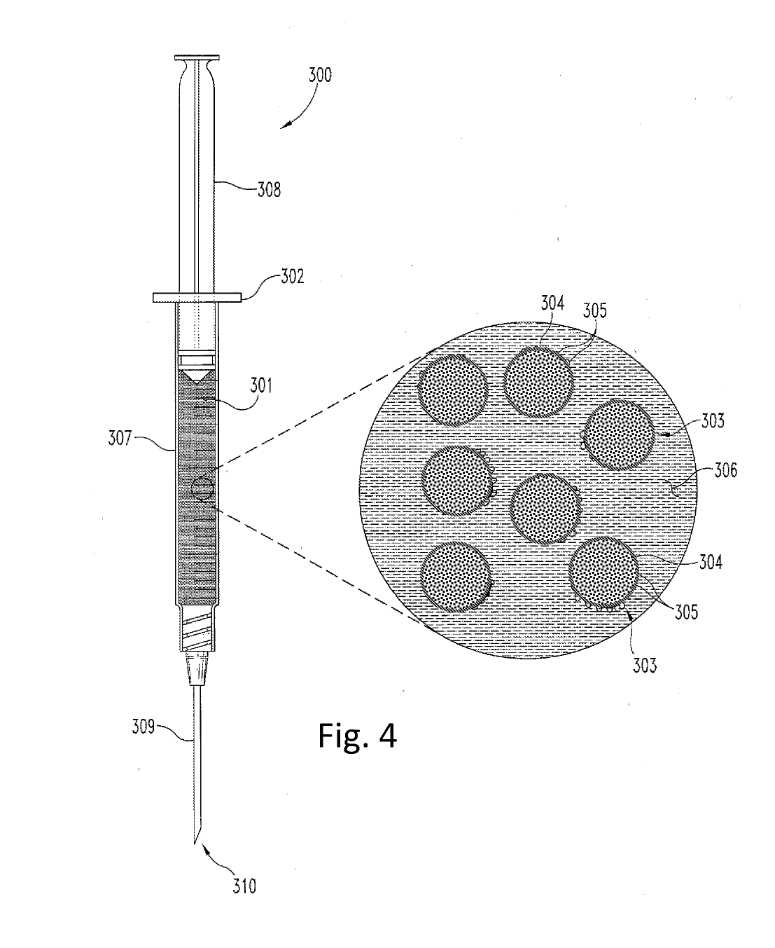

To prepare a cell seeded graft composition, a sheet-form scaffold particle or a composition comprising a population of such particles as described herein can be combined with a cellular preparation. For flowable grafts, the scaffold particle(s) can be suspended in a liquid medium, such as an aqueous medium. Prior to administration, the cells and particle(s) can in some practices be incubated during a cell attachment period, so that cells attach to the particles(s). The size and sheet form the particle(s) provide advantageous suspension and cell attachment characteristics, which are enhanced when a flexible substrate material, such as an extracellular matrix sheet material, is used. For administration to the patient, the cell seeded particle(s) can be loaded in a syringe or other delivery device, and the graft delivered to a tissue targeted for grafting. Illustratively, with reference to FIG. 4, shown is a medical device 300 including a flowable cellular graft composition 301 loaded in a syringe 302. Cellular graft composition 301 includes a plurality of cellularized bodies 303 that include sheet-form scaffold particles 304, as discussed herein, and a population of cells 305 attached to each particle 304. In certain embodiments the cells 305 can form a generally confluent layer of cells covering the matrix particle 304. The cellularized bodies 303 are suspended in a liquid medium 306, such as an aqueous medium optionally containing nutrients for the cells, and which is physiologically compatible with a human or other patient. Cellular graft composition 301 is flowable and received within the barrel 307 of the syringe 302. A plunger 308 is received within barrel 307 and operable upon linear actuation to drive composition 301 through the fluidly coupled needle 309 and out the opening 310 thereof. Medical device 300 can therefore be used to administer the composition 301 into tissues of the patient. In certain preferred embodiments, the target tissues are in need of revascularization and the cellular graft bodies 303 include cells 305 capable of forming blood vessels, for example endothelial cells or endothelial progenitor cells, including in certain embodiments endothelial colony forming cells as discussed herein. Upon injection into the target tissue, the matrix particles 304 will assist in retention of the cells 305 in the targeted region. In particularly preferred embodiments, particles 304 are extracellular matrix particles as described herein.

As disclosed above, in certain embodiments, cellular grafts can be prepared by incubating cells in the presence of the sheet-form particles for a period sufficient for attachment of the cells to the particles. In further embodiments, the cells can be incubated in culture with the particles for a longer period than that needed for cell attachment. In these embodiments, the cells may remodel the scaffold particles, for example depositing extracellular matrix proteins, such as collagen, that are endogenous to the cells, and potentially also resorbing the extracellular matrix proteins, such as collagen, of the scaffold particles. In some forms, culture of the cells in the presence of the scaffold particles will be for a period of time such that at least 1%, at least 5%, or at least 10% of the collagen present in the cellularized bodies 310 is endogenous to the cells. In other forms, higher percentages of the collagen in the cellularized bodies can be endogenous to the cells, for example at least 50%, or in some instances all or essentially all (above 99.5%) of the collagen present in the cellularized bodies 310 is endogenous to the cells. During such culture periods, the number of cells can be expanded and/or, in the case of cells capable of differentiation, at least some of the cells can undergo differentiation. Illustrative culture periods can, for example, be greater than 2 hours, greater than 6 hours, or greater than 12 hours; and, in some embodiments, the culture periods will be in the range of about 12 hours to 72 hours. After culture periods as described above, a composition including the cellularized bodies can be administered to the patient, e.g. to treat a condition as described herein.

In still further embodiments, after an incubation and/or culture period as described herein, cells can be detached from the cellularized bodies, e.g. to create a single cell suspension of the cells. Detachment can be achieved for example by enzymatically treating the cellularized bodies, e.g. with enzymes such as trypsin and/or collagenase. The cells can then be administered to a patient in the form of a single cell suspension, or can be processed into other graft forms (e.g. seeded onto another scaffold or scaffolds) for administration to a patient, for instance to treat a condition as described herein. If desired, after the cells have been detached, remaining portions of the initial sheet-form scaffold particles (when present) can be separated from the cells by filtration or otherwise prior to administration of the single cell suspension or other uses of the cells.

In additional embodiments, sheet-form scaffold particles as described herein can be used as cell growth supports in suspension culture in order to prepare cell conditioned media which can be isolated from the cells for medical, research or other purposes. It has been discovered that culture in the presence of sheet-form scaffold particles can be used to modify the secretome of cells, for example by causing the cells to secrete chemoattractant and/or inflammatory mediator cytokines in greater amounts than they do in corresponding culture in the absence of the sheet-form scaffold particles. Accordingly, embodiments of the invention include processes in which cells are cultured in a medium on cell growth supports comprising sheet-form scaffold particles, and the medium is separated from the cells. The medium can, if needed, be treated to ensure that it is pathogen free, and administered to patients, e.g. to treat conditions as described herein.

Any one or any combination of a wide variety of cell types can be used in cellular graft-related compositions and methods of the invention. For example, the cells can be skin cells, skeletal muscle cells, cardiac muscle cells, lung cells, mesentery cells, or adipose cells. The adipose cells may be from omental fat, properitoneal fat, perirenal fat, pericardial fat, subcutaneous fat, breast fat, or epididymal fat. In certain embodiments, the cells comprise stromal cells, stem cells, or combinations thereof. As used herein, the term “stem cells” is used in a broad sense and includes traditional stem cells, adipose derived stem cells, progenitor cells, preprogenitor cells, reserve cells, and the like. Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells (e.g. derived from blood, dental tissue, skin, uterine tissue, umbilical cord tissue, placental tissue, etc.), hematopoietic stem cells, central nervous system stem cells, peripheral nervous system stem cells, and the like. Additional illustrative cells which can be used include hepatocytes, epithelial cells, Kupffer cells, fibroblasts, neurons, cardiomyocytes, myocytes, chondrocytes, pancreatic acinar cells, islets of Langerhans, osteocytes, myoblasts, satellite cells, endothelial cells, adipocytes, preadipocytes, biliary epithelial cells, regenerative cells, and progenitor cells of any of these cell types.

In some embodiments, the cells incorporated in the cellular grafts are, or include, endothelial progenitor cells (EPCs). Preferred EPCs for use in the invention are endothelial colony forming cells (ECFCs), especially ECFCs with high proliferative potential. Suitable such cells are described for example in U.S. Patent Application Publication No. 20050266556 published Dec. 1, 2005, publishing U.S. patent application Ser. No. 11/055,182 filed Feb. 9, 2005, and U.S. Patent Application Publication No. 20080025956 published Jan. 1, 2008, publishing U.S. patent application Ser. No. 11/837,999, filed Aug. 13, 2007, each of which is hereby incorporated by reference in its entirety. Such ECFC cells can be a clonal population, and/or can be obtained from umbilical cord blood of humans or other animals. Additionally or alternatively, the endothelial colony forming cells have the following characteristics: (a) express the cell surface antigens CD31, CD105, CD146, and CD144; and/or (b) do not express CD45 and CD14; and/or (c) ingest acetylated LDL; and/or (d) replate into at least secondary colonies of at least 2000 cells when plated from a single cell; and/or (e) express high levels of telomerase, at least 34% of that expressed by HeLa cells; and/or (f) exhibit a nuclear to cytoplasmic ratio that is greater than 0.8; and/or (g) have cell diameters of less than about 22 microns. Any combination of some or all of these features (a)-(g) may characterize ECFCs used in the present invention.

In other embodiments, the cells incorporated in the cellular grafts are, or include, muscle derived cells, including muscle derived myoblasts and/or muscle derived stem cells. Suitable such stem cells and methods for obtaining them are described, for example, in U.S. Pat. No. 6,866,842 and U.S. Pat. No. 7,155,417, each of which is hereby incorporated herein by reference in its entirety. The muscle derived cells can express desmin, M-cadherin, MyoD, myogenin, CD34, and/or Bcl-2, and can lack expression of CD45 or c-Kit cell markers.

In still other embodiments, the cells incorporated in the cellular grafts are, or include, stem cells derived from adipose tissue. Suitable such cells and methods for obtaining them are described for example in U.S. Pat. No. 6,777,231 and U.S. Pat. No. 7,595,043, each of which is hereby incorporated herein by reference in its entirety. The cellular population can include adipose-derived stem and regenerative cells, sometimes also referred to as stromal vascular fraction cells, which can be a mixed population including stem cells, endothelial progenitor cells, leukocytes, endothelial cells, and vascular smooth muscle cells, which can be adult-derived. In certain forms, cellular grafts of the present invention can be prepared with and can include adipose-derived cells that can differentiate into two or more of a bone cell, a cartilage cell, a nerve cell, or a muscle cell.

Graft materials and/or cell conditioned media of and prepared in accordance with aspects of the invention can be used in a wide variety of clinical applications to treat damaged, diseased or insufficient tissues, and can be used in humans or in non-human animals. Such tissues to be treated may, for example, be muscle tissue, nerve tissue, brain tissue, blood, myocardial tissue, cartilage tissue, organ tissue such as lung, kidney or liver tissue, bone tissue, arterial or venous vessel tissue, skin tissue, ocular tissue, and others.

In certain embodiments, the grafts or conditioned media can be used to enhance the formation of blood vessels in a patient, for example to alleviate ischemia in tissues. Direct administration of blood vessel-forming cellular grafts, for example grafts containing endothelial colony forming cells or other endothelial progenitor cells, to an ischemic site can enhance the formation of new vessels in the affected areas and improve blood flow or other outcomes. The ischemic tissue to be treated may for example be ischemic myocardial tissue, e.g. following an infarction, or ischemic tissue in the legs or other limbs such as occurs in critical limb ischemia. A cellular graft administered to the ischemic tissue can be a flowable graft material, and in particular an injectable graft material, as disclosed herein.

The grafts or conditioned media can also be used to enhance the healing of partial or full thickness dermal wounds, such as skin ulcers, e.g. diabetic ulcers, and burns. Illustratively, the administration of grafts containing endothelial colony forming cells or other endothelial progenitor cells, or stem cells, or cell conditioned media, to such wounds can enhance the healing of the wounds. These and other topical applications of the grafts or conditioned media are contemplated herein.

In other applications, the grafts or conditioned media can be used to generate or facilitate the generation of muscle tissue at a target site, for example in the treatment of skeletal muscle tissue, smooth muscle tissue, myocardial tissue, or other tissue. Illustratively, cellular grafts of the invention containing muscle derived myoblasts can be delivered, e.g. by injection, into muscle tissue of a sphincter such as a urinary bladder sphincter to treat incontinence.

In still other applications, grafts as described herein can be used for intra-articular injection, or as a building block for engineered tissue.

For the purpose of promoting a further understanding of aspects of the invention and their features and advantages, the following specific Experimental is provided. It will be understood that this Experimental description is illustrative, and not limiting, of aspects of the invention.

### EXPERIMENTAL

### Materials and Methods

### Matrix (SIS Disc) Production

The small intestinal submucosa (SIS) material was obtained from the intestine in a manner that removes all cells, but leaves the naturally fibrous and porous nature of the matrix (Cook Biotech, Inc., USA). The careful processing leaves the complex extracellular matrix available for new cell ingrowth. The thin, yet strong layer of the small intestine from which SIS products are derived possesses a 3-dimensional architecture that allows for intimate cell contact and consists primarily of protein. SIS products are manufactured using a process that minimizes the loss of the natural extracellular matrix components. To assure patient safety, the SIS material undergoes a thorough disinfection, decellularization, and viral inactivation process. As a final step in the process, all SIS products are sterilized by validated sterilization methods. To generate the culture disk matrix, sub-millimeter discs were cut using a punching system that allows for consistent generation of large numbers of discs (see FIG. 1).

### C-URCs Isolation and Primary Culture on SIS Discs

Fully intact uteri were obtained from a local low-cost spay-neuter clinic from female canines that had presented for ovariohysterectomy. The tissues used in this study would have otherwise been discarded as medical waste. Once the samples arrived at the laboratory, the ovaries were removed and discarded then the uterus separated into approximate one gram, full thickness sections.

A one gram sample was then minced to ≦1 mm³ fragments using a sterile scalpel. The chopped tissue was placed into an enzymatic bath and digested for 30 min at 37° C. as described above. Once digestion was complete, the enzymes were neutralized with culture media (DMEM-HG with 10% fetal bovine serum and 0.25 mg/mL amphotericin B, 100 IU/mL penicillin-G, and 100 mg/mL streptomycin), centrifuged at 300×g for 5 min and re-suspended in fresh culture media. The contents were then strained through a 200 μm sterile membrane and plated in a 25 cm² flask. After 14 days of culture, the cells were split as Passage 0 (P0) using TrypZean™ solution (all reagents in this study were obtained from Sigma Chemical, USA, unless otherwise stated) and cell counts and viability were assessed using a standard trypan blue dye exclusion assay and hematocytometer. The resulting cells are termed canine uterine regenerative cells (C-URCs).

SIS discs were conditioned by incubation overnight in complete media. The discs were then plated at 10 cm²/ml into non adhering 24 well plates. Canine URCs were then added to the experimental wells at 7700 cells/cm². Control wells for each of the two plates were also prepared. Cells were incubated with URCs for 9 days at 37° C. and 6% CO₂ with gentle rocking.

Every three days (day 3, 6, and 9) 150 μL of spent media was removed and stored at −20° C. for multiplex analysis (performed at the end of experiment) and replaced with fresh complete media. Media was evaluated for GM-CSF, IL-2, IL-6, IL-7, IL-8, IL-15, IP-10, KC-Like, IL-10, IL-18, MCP-1, and TNF-α.

### Evaluation of Cellular Integrity Following Injection

For these experiments, HeLa cells expressing red fluorescence protein was used (RPF-HeLa). Briefly, trypsinized HeLa cells (2×10⁷) were removed from culture and centrifuged at 300-500 g for 4 min at 22° C. Cells were resuspended in PBS with calcium and magnesium. Using a luer-to-luer syringe connector, 1.5 ml of SIS particulate was mixed with 500 ul of PBS (with calcium/magnesium) by passing it 20 times between syringes. Next, a volume of SIS particulate equal to that of the RFP-HeLa cells in PBS was transferred to a 1 mL syringe, which were then mixed via 2-way luer-to-luer connector with SIS discs by passing between syringes 3-4 times. Approximately 200 μl of the cell-SIS combination was moved into one of the 1 mL syringe, a syringe tip cap affixed to the luer connector, and the syringe placed in an incubator at 37° C.

After a minimum of 30 minutes, the syringe was removed from the incubator, a 23 G needle was attached, and 100 μl of the SIS discs+RFP-HeLa cells was injected into hind limb muscle of a mouse.

### Results

C-URC attached to the SIS ECM disc readily and exhibited good morphology (FIG. 2). Measurement of pro-inflammatory and anabolic cytokines in the resulting cultures indicated levels below detectable parameters while chemoattractant and inflammatory mediator cytokines appeared to be upregulated (FIG. 3). HeLa cells combined with the SIS BioDiscs indicated high viability and stability after 48 hours post injection (FIG. 4).

### Conclusions

SIS ECM discs provide a substrate for cell culture and/or expansion that could provide additional benefits over current scale-up therapeutic systems.
Pro-inflammatory cytokines were below detectable parameters from cells cultured on the SIS ECM Discs, while chemoattractant and inflammatory mediator cytokines appeared upregulated.
The SIS ECM Discs appeared to protect cells upon injection
Celluarized ECM discs have potential as a standalone cell based therapy with enhanced growth factor availability and without the need for trypsinization of cells

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

1. A method for preparing cell growth scaffold particles, comprising:
forcing at least one punch through at least one sheet of cell growth scaffold material to remove from the sheet a sheet-form scaffold particle; and
collecting the sheet-form scaffold particle removed from the sheet in said forcing step.

2. The method of claim 1, also comprising applying tension to said at least one sheet during said forcing.

3. The method of claim 2, wherein said applying tension includes pressing a resilient member against the at least one sheet.

4. The method of claim 2, wherein said pressing occurs during said forcing, and is released during movement of the punch to withdraw the punch from the at least one sheet.

5. The method of claim 3, wherein the resilient member comprises a resilient tubular wall having a leading end defining a perimeter, and wherein said pressing includes pressing the leading end of the tubular wall against the at least one layer.

6. The method of claim 4, wherein during said pressing the perimeter surrounds the punch.

7. The method of claim 1, conducted so as to cell growth scaffold particles constituting at least 40% by weight of the one or more sheets, more preferably at least 50%, and more preferably 50-60%.

8. The method of claim 1, including conducting said forcing step multiple times to create multiple holes in the one or more sheets, wherein the cell growth scaffold particles have been removed to create the holes, and wherein the holes are spaced from one another.

9. The method of claim 7, wherein adjacent ones of the holes are spaced from one another by at least about 0.1 mm.

10. The method of claim 1, wherein said collecting includes gathering the sheet-form supports in a passage in the punch.

11. The method of claim 1, wherein the punch enters the at least one sheet from a first side of the sheet, and wherein said collecting includes discharging the cell growth scaffold particles through and past a second side of the at least one sheet.

12. A method according to claim 1, wherein the at least one sheet includes at least two sheets in a stacked configuration, and preferably wherein the at least one sheet includes two to ten sheets in a stacked configuration.

13. A method according to claim 1, wherein the at least one punch includes at least two punches, and preferably wherein the at least one punch includes two to twenty punches.

14. The method of claim 13, wherein said forcing includes simultaneously forcing the at least two punches, and preferably the two to twenty punches, through the at least one sheet of cell growth scaffold material to remove sheet-form scaffold particles from the sheet.

15. The method of claim 1, wherein the at least one sheet of scaffolding material comprises an extracellular matrix tissue material, and preferably wherein the tissue material retains one or more bioactive agents native to the source tissue of the extracellular matrix tissue material, and more preferably wherein the one or more bioactive agents includes basic fibroblast growth factor (FGF-2), transforming growth factor beta (TGF-beta), epidermal growth factor (EGF), cartilage derived growth factor (CDGF), platelet derived growth factor (PDGF), glycoproteins, proteoglycans, and/or glycosaminoglycans.

16. The method of claim 1, wherein the at least one sheet of scaffolding material comprises extracellular matrix tissue material which is membranous tissue with a sheet structure as isolated from a tissue source.

17. Sheet-form cell growth scaffold particles prepared according to any claim 1.

18. A particulate cell growth scaffold composition, comprising:
a population of sheet-form cell growth scaffold particles, wherein the particles have perimeters defined by cut edges.

19. The composition of claim 18, wherein the cut edges are mechanically-cut edges.

20. The composition of claim 18, wherein the cut edges are free from heat denatured collagen and present exposed cut ends of collagen fibers.

21. The composition of claim 18, wherein the particles have a circular, ovoid or polygonal shape.

22. The composition of claim 18, wherein the scaffold particles comprise an extracellular matrix tissue material, and preferably wherein the tissue material retains one or more bioactive agents native to the source tissue of the extracellular matrix tissue material, and more preferably wherein the one or more bioactive agents includes basic fibroblast growth factor (FGF-2), transforming growth factor beta (TGF-beta), epidermal growth factor (EGF), cartilage derived growth factor (CDGF), platelet derived growth factor (PDGF), glycoproteins, proteoglycans, and/or glycosaminoglycans.

23. The composition of claim 22, wherein the scaffold particles comprise a membranous extracellular matrix tissue material.

24. The composition of claim 18, wherein the scaffold particles incorporate a cell culture medium, blood, or a blood fraction.

25. The composition of claim 18, wherein the scaffold particles are in a dried condition.

26. The composition of claim 18, wherein the scaffold particles are in a lyophilized condition.

27. The composition of claim 18, also comprising cells, and preferably wherein the cells are any one of, or combination of, the cells identified hereinabove.

28. The composition of claim 18, also comprising cells attached to the scaffold particles, and preferably wherein the cells are any one of, or combination of, the cells identified hereinabove

29. The composition of claim 18, wherein the sheet-form scaffold particles have a maximum cross sectional dimension of about 20 microns to about 2000 microns, more preferably about 100 to about 1000 microns, and more preferably about 100 to 500 microns; and preferably also wherein the sheet-form scaffold particles have a sheet thickness less than said maximum cross sectional dimension.

30. A method for preparing a composition, comprising:
incubating cells in suspension in the presence of a composition according to claim 18, so as to cause the cells to attach to the sheet-form scaffold particles.

31. The method of claim 30, also comprising culturing the cells sufficiently to form cellularized bodies in which the cells have deposited extracellular matrix proteins endogenous to the cells in and/or on the sheet-form scaffold particles.

32. The method of claim 31, wherein said culturing is sufficiently conducted that at least 1%, preferably at least 2%, more preferably at least 10%, of the collagen in said cellularized bodies is endogenous to the cells.

33. A method according to claim 30, also comprising detaching the cells from the sheet-form scaffold particles, or from the cellularized bodies.

34. The method of claim 33, also comprising forming a single cell suspension from the cells upon or after said detaching.

35. The method of claim 33, wherein said detaching comprises contacting the sheet-form scaffold particles or cellularized bodies with an enzyme, preferably wherein the enzyme is trypsin and/or collagenase.

36. The method of claim 33, also comprising, after said detaching, separating remnants of said sheet-form scaffold particles from said cells.

37. The method of claim 30, also comprising collecting a liquid medium which has been conditioned during said incubating and/or said culturing.

38. The method of claim 37, also comprising sterilizing said liquid medium.

39. A method for treating a patient, comprising administering to the patient cell growth scaffold particles prepared according to claim 1, a particulate cell growth scaffold composition according to claim 18, or a composition prepared according to claim 30.

40. A method according to claim 39, wherein said administering is by injection.

41. A method according to claim 39, for treatment of treat damaged, diseased or insufficient tissues, including any of those identified hereinabove.
